(19) United States
(12) Patent Application Publication
(10) Pub. No.: US 2020/0268670 A1
(43) Pub. Date: Aug. 27, 2020

(54) Trans-Catheter Local Immunotherapy for Solid Tumors

(71) Applicant: Endobar Solutions LLC, Orangeburg, NY (US)

(72) Inventors: Nicholas KIPSHIDZE, New York, NY (US); Zurab KAKABADZE, Tbilisi (GE)

(73) Assignee: Endobar Solutions LLC, Orangeburg, NY (US)

(21) Appl. No.: 16/797,165

(22) Filed: Feb. 21, 2020

Related U.S. Application Data
(60) Provisional application No. 62/811,807, filed on Feb. 28, 2019.
(60) Provisional application No. 62/809,149, filed on Feb. 22, 2019.

(51) Int. Cl. A61K 9/16; A61M 25/10; A61K 35/17; A61K 9/00

(52) U.S. Cl. CPC A61K 9/1676; A61K 9/0019; A61K 35/17; A61M 25/10

(57) ABSTRACT

Tumors can be treated by introducing a first embolization agent (e.g., small embolic beads) into a blood vessel that supplies blood to the tumor, then introducing a therapeutic substance into the blood vessel at a position that is proximal with respect to the first embolization agent, and then introducing a second embolization agent (e.g., large embolic beads) into the blood vessel at a position that is proximal with respect to at least a portion of the therapeutic substance. The first embolization agent prevents complete systemic release of the substance, and the second embolization agent prevents retrograde washout of the substance. In some embodiments, the first embolization agent is omitted, and the second embolization agent impedes systemic release by impeding the forward flow of blood. In some embodiments, the therapeutic substance is an immunotherapy substance (e.g., CAR T-cells).

### CROSS REFERENCE TO RELATED APPLICATIONS

This application claims the benefit of U.S. Provisional Applications 62/809,149 (filed Feb. 22, 2019) and 62/811,807 (filed Feb. 28, 2019) each of which is incorporated herein by reference in its entirety.

### BACKGROUND

Most treatments for advanced malignancies, including immunotherapies, are administered systemically.

Equivalent or improved results can be obtained with less toxicity by locally injecting a therapeutic substance directly to the tumor or draining lymph nodes. Local delivery can prevent high levels of the drug in systemic circulation, thus reducing toxicity. Furthermore, delivering higher concentrations of immunotherapeutic agents locally at the injection site should induce a more robust, systemic antitumor immune response against the most immunogenic neoantigens within the tumor. A goal of this approach is to eradicate or shrink the tumor at the injection site(s) and also to create a “danger signal” that induces a systemic CD8+ tumor-infiltrating lymphocyte (TIL) response that targets cancer throughout the body (a process known as an “abscopal effect).

Local therapy may be performed via direct injections to a tumor and/or nodes. But this approach has a number of limitations. For example, lesions need to be above a certain size threshold and relatively accessible, which can be a problem if repeated treatment is necessary. Anatomical distance usually determines an injection site for local immunotherapy; however, injecting a primary tumor may result in better response due to mutations shared with metastases. The optimal injection volume and the best delivery methods are often unknown, while bleeding or infection present associated risks.

### SUMMARY OF THE INVENTION

One aspect of the invention is directed to a first method of treating a tumor with a substance. The first method comprises introducing a first embolization agent into a blood vessel that supplies blood to the tumor; introducing the substance into the blood vessel after the first embolization agent has been introduced into the blood vessel, at a position that is proximal with respect to the first embolization agent; and introducing a second embolization agent into the blood vessel after the substance has been introduced into the blood vessel, at a position that is proximal with respect to at least a portion of the substance. The first embolization agent operates to block outflow and prevent complete systemic release of the substance, and the second embolization agent operates to block inflow and prevent retrograde washout of the substance.

In some instances of the first method, the first embolization agent comprises first embolic beads, the second embolization agent comprises second embolic beads, and the second embolic beads are larger than the first embolic beads. Optionally, in these instances, the first embolic beads have diameters less than or equal to 100 μm, and the second embolic beads have diameters greater than or equal to 200 μm.

In some instances of the first method, the introducing of the first embolization agent into the blood vessel is accomplished by inflating a balloon to block the blood vessel and introducing the first embolization agent at a position that is distal with respect to the inflated balloon.

In some instances of the first method, the introducing of the substance into the blood vessel is accomplished at a pressure that is sufficient to overcome a region of high pressure in a vicinity of the tumor. In some instances of the first method, the introducing of the substance into the blood vessel is accomplished at a pressure between 10 and 200 mmHg relative to the local pressure.

In some instances of the first method, the introducing of the substance into the blood vessel is controlled to maintain laminar flow and to prevent non target and systemic release of the substance. In some instances of the first method, the substance comprises an immunotherapy substance. In some instances of the first method, the substance comprises CAR T-cells.

Another aspect of the invention is directed to a second method of treating a tumor with a substance. The second method comprises introducing a catheter into a blood vessel that supplies blood to the tumor; inflating a balloon to block the blood vessel; introducing the substance into the blood vessel at a position that is distal with respect to the inflated balloon at a pressure that is sufficient to overcome a region of high pressure in a vicinity of the tumor; and introducing an embolization agent into the blood vessel after the substance has been introduced into the blood vessel, at a position that is proximal with respect to at least a portion of the substance. The embolization agent acts to prevent retrograde washout of the substance and impede systemic release by impeding the forward flow of blood.

In some instances of the second method, the embolization agent comprises embolic beads with diameters greater than or equal to 200 μm. In some instances of the second method, the introducing of the substance into the blood vessel is accomplished at a pressure between 10 and 200 mmHg relative to the local pressure. In some instances of the second method, the introducing of the substance into the blood vessel is controlled to maintain laminar flow and to prevent non target and systemic release of the substance. In some instances of the second method, the substance comprises an immunotherapy substance. In some instances of the second method, the substance comprises CAR T-cells.

Another aspect of the invention is directed to a third method of treating a tumor with a substance. The third method comprises introducing a catheter into a blood vessel that supplies blood to the tumor; inflating a balloon to block the blood vessel; and introducing a liquid in which a plurality of substance-eluting beads are suspended into the blood vessel at a position that is distal with respect to the inflated balloon at a pressure that is sufficient to overcome a region of high pressure in a vicinity of the tumor.

In some instances of the third method, the introducing of the liquid into the blood vessel is accomplished at a pressure between 10 and 200 mmHg relative to the local pressure. In some instances of the third method, the introducing of the liquid into the blood vessel is controlled to maintain laminar flow and to prevent non target and systemic release of the substance. In some instances of the third method, the substance-eluting beads comprise an immunotherapy substance. In some instances of the third method, the substance-eluting beads comprise CAR T-cells. In some instances of the third method, the substance-eluting beads primarily comprise agarose.

### BRIEF DESCRIPTION OF THE DRAWINGS

FIG. 1 is a schematic representation of a network of blood vessels that supply blood to a target location.

FIG. 2 is a flowchart of a first process for delivering a therapeutic substance to the blood vessels that supply blood to a target location.

FIG. 3 is a schematic representation of introducing a balloon catheter to the blood vessels that supplies blood to the tumor.

FIG. 4 depicts the same catheter after the balloon has been inflated.

FIG. 5 depicts the introduction of small embolic beads into the blood vessels.

FIG. 6 depicts the introduction of a therapeutic substance into the blood vessels at a position that is proximal with respect to the small embolic beads.

FIG. 7 depicts the introduction of large embolic beads into the blood vessel at a position that is proximal with respect to at least a portion of the therapeutic substance.

FIG. 8 is a flowchart of a second process for delivering a therapeutic substance to the blood vessels that supply blood to a target location.

FIG. 9 is a schematic representation of introducing the therapeutic substance into the blood vessel.

FIG. 10 depicts the introduction of large embolic beads into the blood vessel at a position that is proximal with respect to at least a portion of the therapeutic substance.

FIG. 11 is a flowchart of a third process for delivering a therapeutic substance to the blood vessels that supply blood to a target location.

FIG. 12 depicts the introduction of substance-eluting beads into the blood vessels.

Various embodiments are described in detail below with reference to the accompanying drawings, wherein like reference numerals represent like elements, and wherein optional steps are represented by dashed lines.

### DESCRIPTION OF THE PREFERRED EMBODIMENTS

The embodiments described herein use an alternative approach for local delivery of substances (including but not limited to immunotherapy substances) to the vicinity of a tumor. More specifically, local delivery of therapeutic substances is accomplished via blood vessels that feed the tumor using a trans-catheter approach.

In some embodiments, trans-catheter local immunotherapy is combined with direct embolization using micro beads.

One issue that must be addressed in the context of delivering therapeutic substances to the vicinity of the tumor is that interstitial fluid pressure (IFP) is elevated in many solid tumors. And this elevation in IFP has been associated with low efficacy of trans-arterial chemotherapy and/or radioembolization, because the elevated IFP makes it harder to deliver the material to the desired location (i.e., the vicinity of the tumor).

A set of experiments was performed intraoperatively in 5 patients with hepatocellular carcinoma undergoing resection (i.e., partial hepatectomy) of a liver tumor to determine the degree of IFP elevation in liver tumors and to evaluate the influence of modulating tumor-feeding arterial pressure on IFP. IFP was measured directly in the body of the tumor, healthy tissue, and arterial pressure in hepatic arteries before and after the blood supply to the relevant anatomy was blocked by clipping of the hepatic artery or its branches.

Interstitial pressure of the normal hepatic parenchyma was measured while a 21 gauge needle was being advanced to the tumor under visual control. The pressure was then obtained when the needle entered the periphery and/or body of the tumor. The blood pressure of the hepatic arteries was also obtained at the time of the IFP measurement, and the mean arterial pressure was calculated. More specifically, interstitial pressure in tumors was measured during surgery both before and after the clipping of the tumor feeding artery. To measure, IFP, a 21 gauge needle with a 2 mm side hole at a distance of 5 mm from the tip was used. To measure the pressure of the needle and the needle-connected tube, which was filled with sterile heparinized saline, it was connected to a pressure sensor and an electronic data collection and registration system. The needle was inserted into three areas of the tumor (1 point-12 hours, 2 point-8 hours and 3 point-4 hours). IFP was recorded at all three points of the tumor. After clipping of the tumor feeding artery, pressure measurements in the body of tumor were conducted in the same fashion. The measurements were performed at 5, 10 and 15 min after artery clipping. The liver was resected with the removal of the hepatocellular carcinoma.

The results of the experiments were as follows. Tumor IFP was higher than in healthy tissue by 10-30 mm Hg. But after the relevant arteries were clipped, the IFP decreased by 27.3%±5.2 (p<0.01). IFP reduction post-clipping significantly correlated to the tumor feeding hepatic arteries mean arterial pressure post-procedure (r=0.613, p=<0.01) Based on these experiments, the inventor concluded that there is clear relationship between IFP and intra-arterial pressure of the tumor feeding arteries, and that the IFP is reduced when blood flow in the tumor feeding arteries is blocked.

In the context of delivering therapeutic substances via blood vessels that feed the tumor using a trans-catheter approach, clipping the artery is not a practical solution for blocking an artery in order to reduce the IFP, because the clipping would interfere with the catheter that is being used to deliver the therapeutic substance. So instead, the embodiments described herein use a balloon catheter configured so that the balloon can selectively block the blood flow through the artery. In these embodiments, the catheter is introduced into a blood vessel that supplies blood to the tumor and the balloon is inflated to block the blood vessel, which results in a reduction of the IFP.

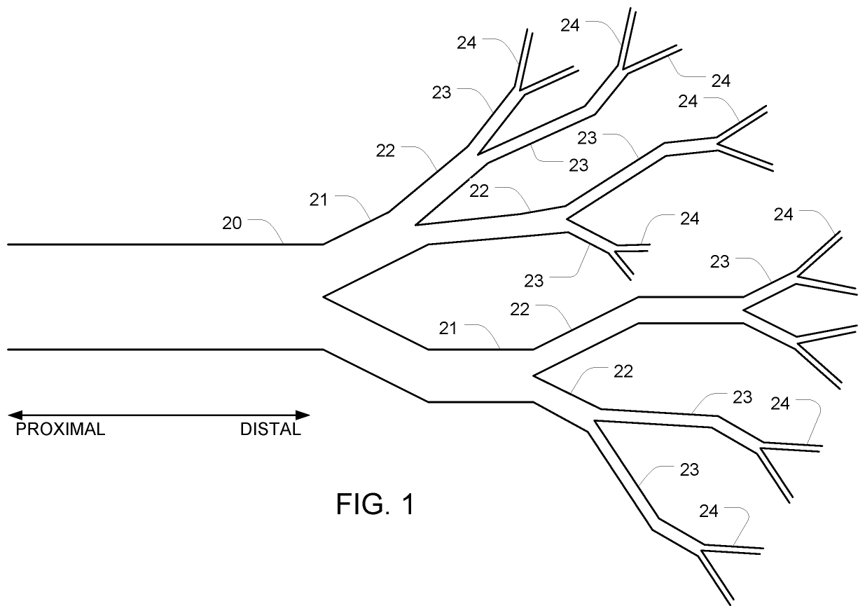

FIG. 1 is a schematic representation of a network of blood vessels that supply blood to a target location (e.g., to a tumor or other part of the body that has a high IFP). The network includes a first artery 20 that is large enough to be accessed by a catheter. Smaller arteries 21 branch off from the first artery 20; still smaller arteries 22 branch off from the arteries 21; still smaller arteries 23 branch off from the arteries 22; and still smaller arteries 24 branch off from the arteries 23.

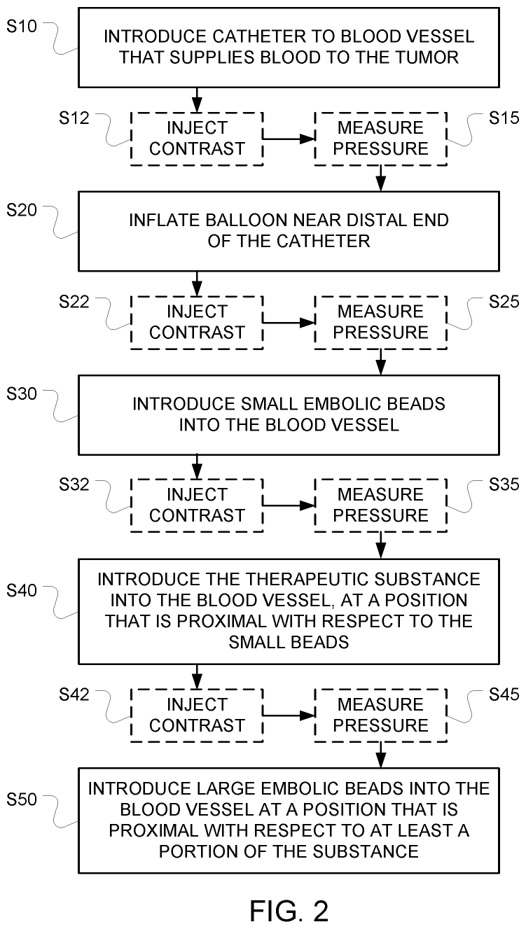

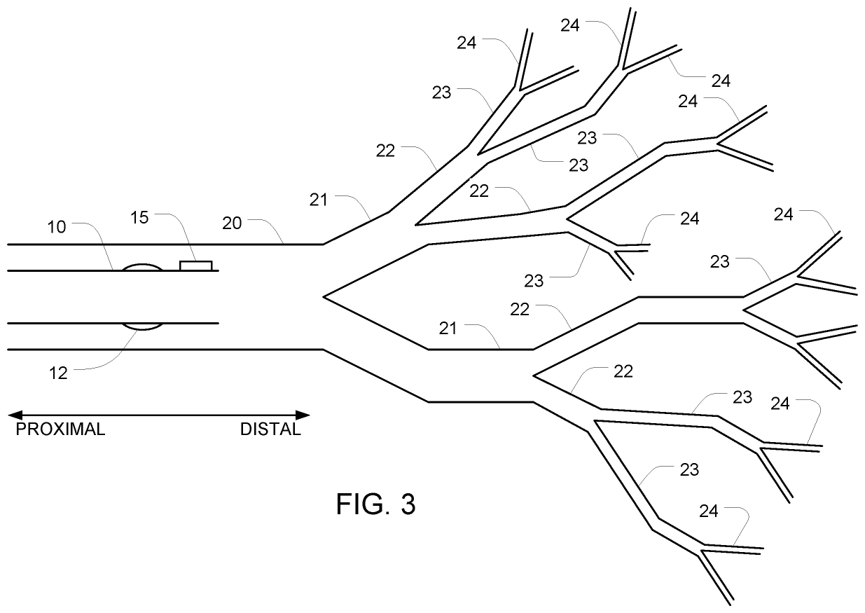

FIG. 2 is a flowchart of a first process for delivering a therapeutic substance to the blood vessels that supply blood to a target location (e.g., a tumor). First, in step S10, a catheter is introduced to the blood vessel that supplies blood to the tumor. The end result of this step is depicted in FIG. 3, which shows the distal end of the catheter 10 positioned in the first artery 20. The catheter 10 has a balloon 12 disposed near the distal end of the catheter, and this balloon is configured so that inflation of the balloon will block the first artery 20. Optionally, a pressure sensor 15 may be positioned on the catheter 10, distally with respect to the balloon 12. Optionally, in step S12, contrast agent may be injected via the catheter to verify the position of the catheter within the blood vessel. Optionally, in step S15, the pressure sensor 15 may be used to make a pressure measurement.

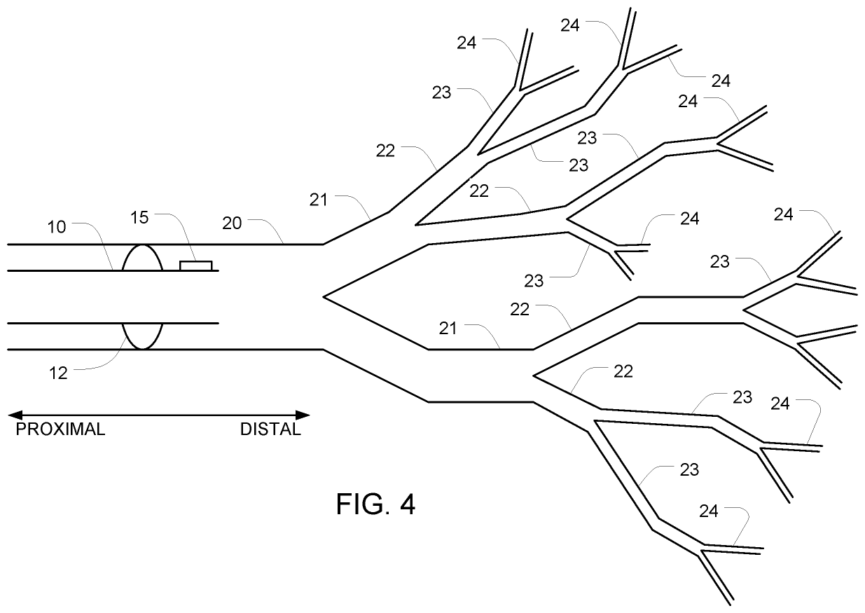

Subsequently, in step S20, the balloon 12 is inflated, and the end result of this step is depicted in FIG. 4. Inflation of the balloon may be accomplished using any of a variety of conventional approaches that will be appreciated by persons skilled in the relevant art. The balloon 12 is inflated to the point where it blocks the blood vessel, which results in a reduction of the IFP. The reduction in IFP is advantageous because it makes it easier to deliver the therapeutic substance to the desired location in subsequent steps. Optionally, in step S22, contrast agent may be injected via the catheter to verify that the balloon has achieved a complete seal. Optionally, in step S25, the pressure in the portion of the blood vessel 20 that is distal with respect to the balloon 12 can be measured in real time using the pressure sensor 15.

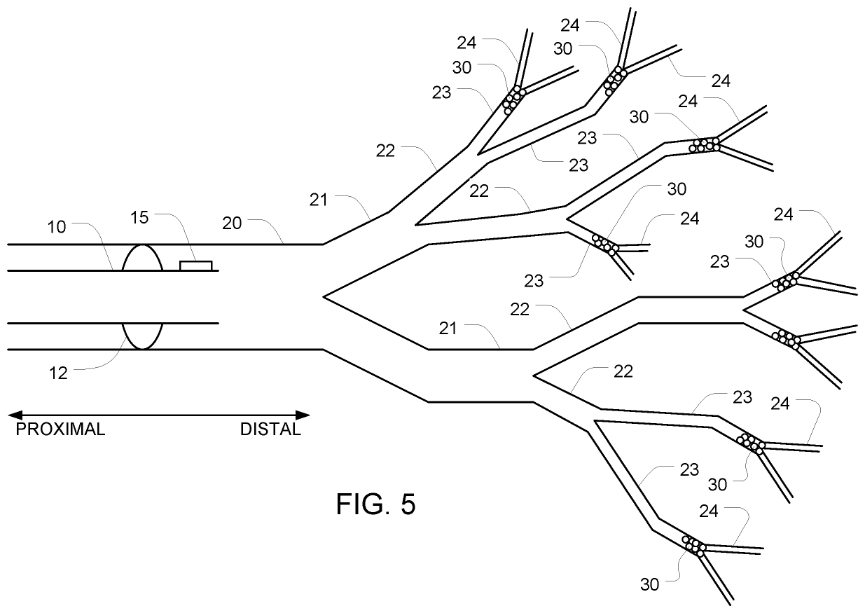

Subsequently, in step S30, a first quantity of small embolic beads 30 is introduced via the catheter 10 into the blood vessel 20 that supplies blood to the tumor at a position that is distal with respect to the inflated balloon. Introduction of the small embolic beads 30 may be implemented manually using a syringe or automatically using a suitable pump (e.g., a peristaltic pump), the operation of which is controlled by a controller to deliver a desired quantity of small embolic beads 30. When a pressure measurement was made in step S25, the pump should be set to a pressure that is sufficient to overcome the pressure measured in step S25. These small embolic beads 30 will travel downstream through the network of blood vessels 21-24 until the blood vessels are small enough to prevent the embolic beads 30 from travelling any further, as depicted in FIG. 5. This will occlude arterioles at the target site and thereby prevent complete outflow and systemic release of the therapeutic substance that is introduced in step S40.

In some preferred embodiments, the small embolic beads 30 have diameters between 5 and 100 μm, and in some preferred embodiments, the small embolic beads 30 have diameters between 10 and 100 μm. In some embodiments, the small embolic beads 30 are echolucent, in which case the position of the beads can be visualized using ultrasound imaging. In some embodiments, the small embolic beads 30 are radio-opaque, in which case the position of the beads can be visualized using CT and/or fluoro imaging. In some embodiments, the small embolic beads 30 may be drug-coated.

In alternative embodiments, a different first embolization agent is used in place of the small embolic beads depicted in FIG. 5. Examples of suitable materials that may be used for the first embolization agent include but are not limited to organic embolization agents (e.g. autologous clot, fibrin, liquid collagen, thrombin, fibrinogen), sclerosants, alcohols, polymerising substances, histoacryl, detergents (e.g., fibrovein, ethoxysclerol), antibiotics (e.g. doxocycllin, bleomycin), precipitating substances (e.g., Onyx®), ethylene vinyl alcohol copolymers, N-butyl cyanoacrylate, microparticles, gelfoam, and polyvinyl alcohol particles.

Optionally, in step S32, contrast agent may be injected via the catheter to verify that the small embolic beads 30 have occluded the relevant blood vessel. If the occlusion is insufficient, additional small embolic beads 30 may be introduced via the catheter 10. Optionally, in step S35, the pressure in the portion of the blood vessel 20 that is distal with respect to the balloon 12 can be measured in real time using the pressure sensor 15.

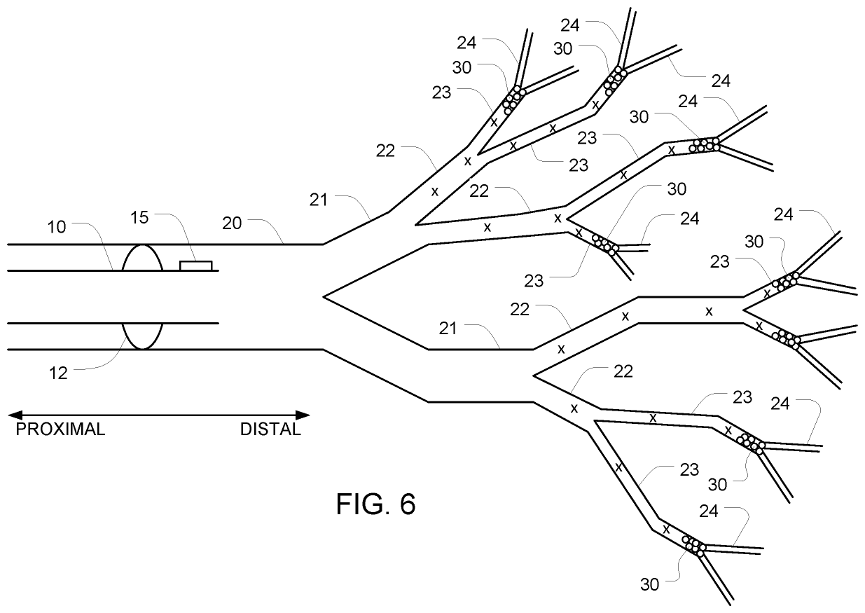

Subsequently, in step S40, while the balloon 12 remains inflated, the therapeutic substance is introduced via the catheter 10 into the blood vessel 20 at a position that is proximal with respect to the first quantity of small embolic beads 30 (or other embolization agent). This is depicted in FIG. 6, in which “X” represents the therapeutic substance. In some preferred embodiments, the introducing of the substance into the blood vessel is accomplished at a pressure that is sufficient to overcome the region of high IFP that naturally occurs in the vicinity of the tumor (which, as explained above, will have already been reduced to some extent by the inflation of the balloon 12). When a pressure measurement was made in step S35, the pump should be set to a pressure that is sufficient to overcome the pressure measured in step S35. In some preferred embodiments, the delivery of the therapeutic substance is accomplished at a pressure between 10 and 200 mmHg relative to the local pressure. In some preferred embodiments, it is accomplished at a pressure between 40 and 200 mmHg relative to the local pressure. Introduction of the therapeutic substance may be implemented manually using a syringe or automatically using a suitable pump (e.g., a peristaltic pump), the operation of which is controlled by a controller to deliver a desired quantity of the therapeutic substance.

The delivery of the therapeutic substance occurs while the balloon 12 is inflated, which causes a reduction in the intratumoral pressure. The reduced pressure advantageously facilitates delivery of the therapeutic substance to the tumor site in a more precise fashion. Preferably, the therapeutic substance is introduced at a flow rate that is low enough to maintain laminar flow and prevent non target and systemic release of the therapeutic substance.

In some embodiments, the therapeutic substance X comprises drug-eluting beads. In some embodiments, the therapeutic substance X comprises an immunotherapy compound. In some embodiments, the therapeutic substance comprises CAR T-cells and/or other cell materials or injectable liquid polymeric and natural material to serve as depot for cells to prolong residence of substances in the tumor body.

Optionally, in step S42, contrast agent may be injected via the catheter to verify that the therapeutic agent is in position. Optionally, in step S45, the pressure in the portion of the blood vessel 20 that is distal with respect to the balloon 12 can be measured in real time using the pressure sensor 15.

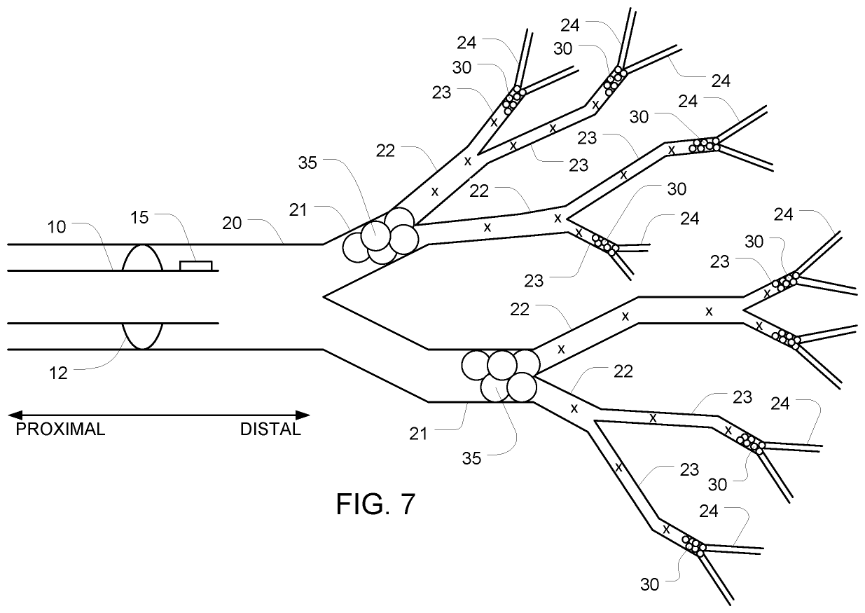

Subsequently, in step S50, while the balloon 12 remains inflated, a second quantity of large embolic beads 35 is introduced via the catheter 10 into the blood vessel 20, at a position that is proximal with respect to at least a portion of the therapeutic substance X. This is depicted in FIG. 7. The large embolic beads 35 will travel downstream through the network of blood vessels 21-24 until the blood vessels are small enough to prevent them from travelling any further. The large embolic beads 35 are larger than the small embolic beads 30, and these large embolic beads 35 prevent retrograde washout of the therapeutic substance X. In some preferred embodiments, the small embolic beads 30 have diameters less than or equal to 100 μm, and the large embolic beads 35 have diameters greater than or equal to 200 μm. Introduction of the large embolic beads 35 may be implemented manually using a syringe or automatically using a suitable pump (e.g., a peristaltic pump), the operation of which is controlled by a controller to deliver a desired quantity of large embolic beads 35. When a pressure measurement was made in step S45, the pump should be set to a pressure that is sufficient to overcome the pressure measured in step S45. In some embodiments, the large embolic beads 35 are echolucent, in which case the position of the beads can be visualized using ultrasound imaging. In some embodiments, the large embolic beads 35 are radio-opaque, in which case the position of the beads can be visualized using CT imaging.

In alternative embodiments, a different second embolization agent is used in place of the large embolic beads 35 depicted in FIG. 7. Examples of suitable materials that may be used for the second embolization agent include but are not limited organic embolization agents (e.g. autologous clot, fibrin, liquid collagen, thrombin, fibrinogen), sclerosants, alcohols, polymerising substances, histoacryl, detergents (e.g., fibrovein, ethoxysclerol), antibiotics (e.g. doxocycllin, bleomycin), precipitating substances (e.g., Onyx®), ethylene vinyl alcohol copolymers, N-butyl cyanoacrylate, particles, gelfoam, polyvinyl alcohol particles, detachable coils, active coils, detachable balloons, and embolic plugs.

In the embodiments depicted above in connection with FIG. 2, the therapeutic substance X is bounded on both sides by embolic beads 30, 35. More specifically, the smaller embolic beads 30 are positioned distally with respect to the therapeutic substance X and the larger embolic beads 35 are positioned proximally with respect to the therapeutic substance V. These two sets of beads 30, 35 (or other embolization agents) operate together to capture the therapeutic substance X at the desired location within the body.

Advantageously, this approach is an improvement with respect to conventional transcatheter delivery of substances because it minimizes non-target delivery, overcomes problems introduced by the high-pressure environment within a tumor, and avoids washout of the therapeutic substance subsequent to its delivery. Optionally, the entire procedure depicted in FIG. 2 (or portions thereof) may be done under fluoro control with contrast injections or CT and ultrasound control.

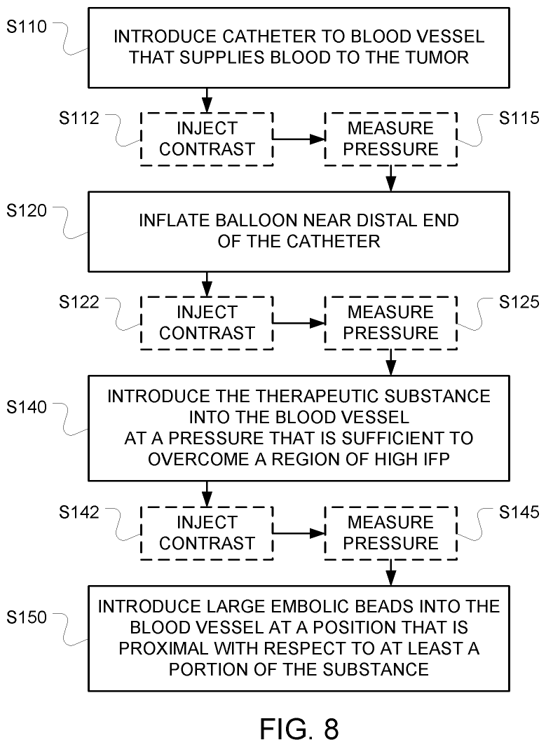

FIG. 8 is a flowchart of a second process for delivering a therapeutic substance to the blood vessels that supply blood to a target location (e.g., a tumor). Steps S110-S125 are identical to steps S10-S25, respectively, in the FIG. 2 embodiment discussed above; and the end results of steps S110 and S120 are depicted in FIGS. 3 and 4, respectively.

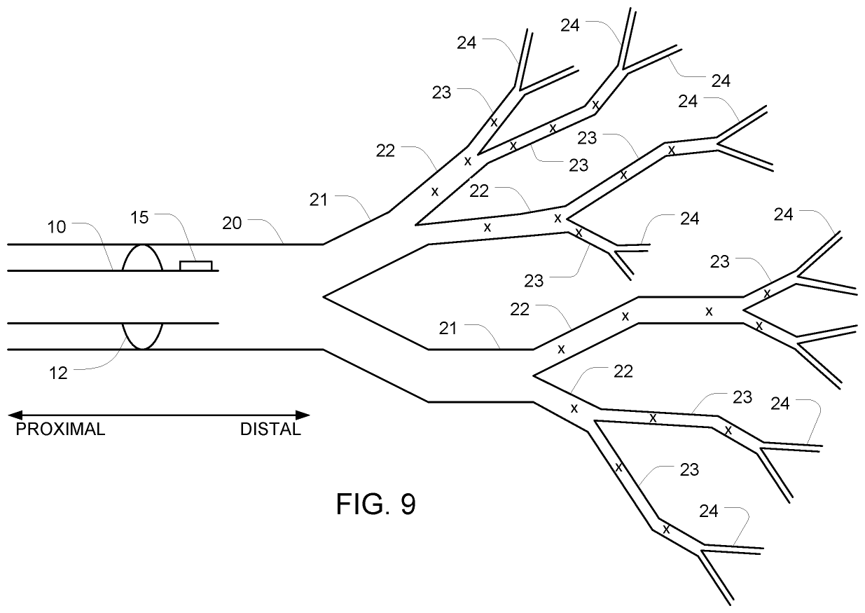

Subsequently, in step S140, while the balloon 12 remains inflated, the therapeutic substance is introduced via the catheter 10 into the blood vessel 20. This is depicted in FIG. 9, in which “X” represents the therapeutic substance. The introducing of the substance into the blood vessel is accomplished at a pressure that is sufficient to overcome the region of high IFP that naturally occurs in the vicinity of the tumor (which, as explained above, will have already been reduced to some extent by the inflation of the balloon 12). When a pressure measurement was made in step S125, the pump should be set to a pressure that is sufficient to overcome the pressure measured in step S125. In some preferred embodiments, the delivery of the therapeutic substance is accomplished at a pressure between 10 and 200 mmHg relative to the local pressure. In some preferred embodiments, it is accomplished at a pressure between 40 and 200 mmHg relative to the local pressure. Introduction of the therapeutic substance may be implemented manually using a syringe or automatically using a suitable pump (e.g., a peristaltic pump), the operation of which is controlled by a controller to deliver a desired quantity of the therapeutic substance.

The delivery of the therapeutic substance occurs while the balloon 12 is inflated, which causes a reduction in the intratumoral pressure. The reduced pressure advantageously facilitates delivery of the therapeutic substance to the tumor site in a more precise fashion. Preferably, the therapeutic substance is introduced at a flow rate that is low enough to maintain laminar flow and prevent non target and systemic release of the therapeutic substance.

In some embodiments, the therapeutic substance X comprises drug-eluting beads. In some embodiments, the therapeutic substance X comprises an immunotherapy compound. In some embodiments, the therapeutic substance comprises CAR T-cells and/or other cell materials or injectable liquid polymeric and natural material to serve as depot for cells to prolong residence of substances in the tumor body.

Optionally, in step S142, contrast agent may be injected via the catheter to verify that the therapeutic agent is in position. Optionally, in step S145, the pressure in the portion of the blood vessel 20 that is distal with respect to the balloon 12 can be measured in real time using the pressure sensor 15.

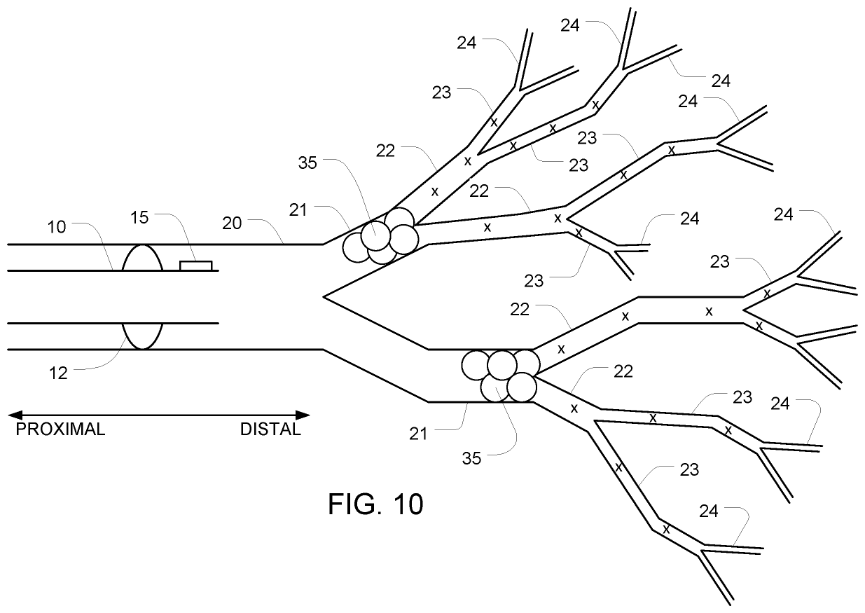

Subsequently, in step S150, while the balloon 12 remains inflated, a quantity of large embolic beads 35 is introduced via the catheter 10 into the blood vessel 20, at a position that is proximal with respect to at least a portion of the therapeutic substance X. This is depicted in FIG. 10. The large embolic beads 35 will travel downstream through the network of blood vessels 21-24 until the blood vessels are small enough to prevent them from travelling any further. The large embolic beads 35 prevent retrograde washout of the therapeutic substance X. In addition, because the large embolic beads 35 will impede the forward flow of blood through the blood vessels 21-24, the large embolic beads 35 will impede the exit of the therapeutic substance X from the vicinity of the tumor in the direction of forward blood flow. As a result, the therapeutic substance X will remain in the vicinity of the tumor for longer than it would have in the absence of the large embolic beads 35. In some preferred embodiments, the large embolic beads 35 have diameters greater than or equal to 200 μm. Introduction of the large embolic beads 35 may be implemented manually using a syringe or automatically using a suitable pump (e.g., a peristaltic pump), the operation of which is controlled by a controller to deliver a desired quantity of large embolic beads 35. When a pressure measurement was made in step S145, the pump should be set to a pressure that is sufficient to overcome the pressure measured in step S145. In some embodiments, the large embolic beads 35 are echolucent, in which case the position of the beads can be visualized using ultrasound imaging. In some embodiments, the large embolic beads 35 are radio-opaque, in which case the position of the beads can be visualized using CT imaging.

In alternative embodiments, a different embolization agent is used in place of the large embolic beads 35 depicted in FIG. 10. For example, any of the alternative embolization agents discussed above in connection with FIG. 7 may be used in this FIG. 8-10 embodiment as well. Optionally, the entire procedure depicted in FIG. 8 (or portions thereof) may be done under fluoro control with contrast injections or CT and ultrasound control.

Advantageously, this approach minimizes non-target delivery, overcomes problems introduced by the high-pressure environment within a tumor, and reduces washout of the therapeutic substance subsequent to its delivery.

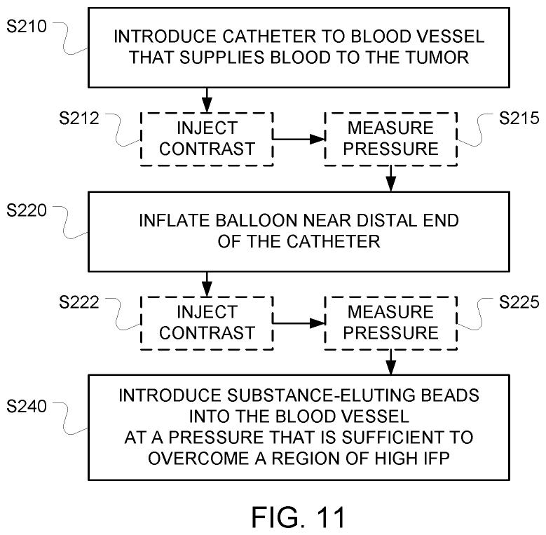

FIG. 11 is a flowchart of a third process for delivering a therapeutic substance to the blood vessels that supply blood to a target location (e.g., a tumor). Steps S210-S225 are identical to steps S10-S25, respectively, in the FIG. 2 embodiment discussed above; and the end results of steps S210 and S220 are depicted in FIGS. 3 and 4, respectively.

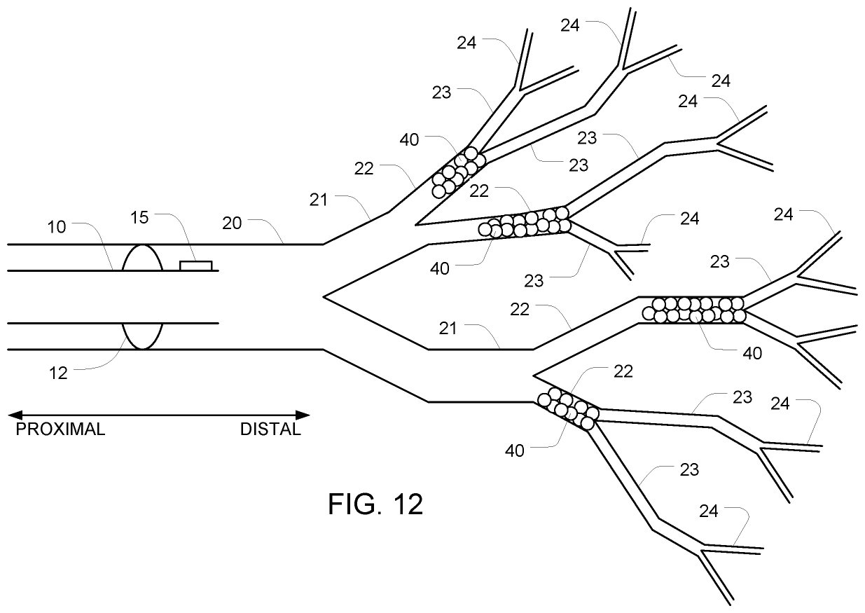

Subsequently, in step S240, a liquid carrier that includes a quantity of substance-eluting beads 40 is introduced into the blood vessel 20 that supplies blood to the tumor via the catheter 10 at a position that is distal with respect to the inflated balloon 12. The substance-eluting beads 40 will travel downstream through the network of blood vessels 21-24 until the blood vessels are small enough to prevent them from travelling any further. The end result of this step S240 is depicted in FIG. 12. This introduction of the substance-eluting beads 40 should be implemented at a pressure that is sufficient to overcome the region of high IFP (which, as explained above, will have already been reduced to some extent by the inflation of the balloon 12). When a pressure measurement was made in step S225, the pump should be set to a pressure that is sufficient to overcome the pressure measured in step S225. In some preferred embodiments, this is accomplished at a pressure between 10 and 200 mmHg relative to the local pressure. In some preferred embodiments, it is accomplished at a pressure between 40 and 200 mmHg relative to the local pressure. In some preferred embodiments, the substance-eluting beads have diameters between 10 and 500 μm. Introduction of the liquid carrier and the substance-eluting beads 40 may be implemented manually using a syringe or automatically using a suitable pump (e.g., a peristaltic pump), the operation of which is controlled by a controller to deliver a desired quantity of substance-eluting beads 40.

Optionally, the substance-eluting beads 40 may use hydrogel and/or nanoparticles. For example, the substance-eluting beads 40 may be made by applying soybean lecithin to entrap hydrophilic bone morphogenic protein-2 into nanoporous poly(lactide-co-glycolide)-based microspheres. Other examples of materials that are suitable for performing the substance-eluting beads 40 include but are not limited to silicon-based hydrogels; PEG-based polymers; nanoparticle-containing hydrogels; hydrogels containing cyclodextrins (CDs); hydrophilic polymers or poly ethylene glycol (PEG) provides water solubility to hydrogels, other polymers like as poly lactic acid (PLA), polyε-caprolactone (PCL), polypropylene oxide (PPO), polyd,l-lactide-co-glycolide (PLGA) and polyε-caprolactone-co-d,l-lactide (PCLA); ultra-thermosensitive hydrogel; hydrogels with different systems, namely, emulsions, vesicular (including micelles, liposomes and nanocapsules) and particulate systems (including mainly solid lipid micro and nanoparticles, nanostructured lipid carriers and lipid drug conjugates); biocompatible hydrogel, composed of the copolymer poly(N-isopropylamide-co-n-butyl methacrylate) [P(NIPAAm-co-BMA)] and PEG; A polyethylenimine (PEI)-based hydrogel; supramolecular hydrogels; DNA-hydrogels; bio-inspired hydrogels; and multi-functional and stimuli-responsive hydrogels.

The delivery of the liquid carrier that includes the substance-eluting beads 40 occurs while the balloon 12 is inflated, which causes a reduction in the intratumoral pressure. The reduced pressure advantageously facilitates delivery of the substance-eluting beads to the tumor site in a more precise fashion. Preferably, the liquid carrier that includes the substance-eluting beads is introduced at a flow rate that is low enough to maintain laminar flow and prevent non target and systemic release of the therapeutic substance.

In some embodiments, the substance-eluting beads are primarily made from agarose. In some embodiments, the substance-eluting beads comprise an immunotherapy compound. In some embodiments, the substance-eluting beads comprise CAR T-cells. Optionally, the entire procedure depicted in FIG. 11 (or portions thereof) may be done under fluoro control with contrast injections or CT and ultrasound control.

Advantageously, this approach is an improvement with respect to conventional transcatheter delivery of substances because it minimizes non-target delivery, overcomes problems introduced by the high-pressure environment within a tumor, and avoids washout of the therapeutic substance subsequent to its delivery.

Notably, in any of the embodiments described above, pressure-driven balloon-assisted embolization may be an effective tool that facilitates improved penetration of anticancer therapeutic agents and overcomes barriers to transcapillary transport.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of treating a tumor with a substance, the method comprising:
introducing a first embolization agent into a blood vessel that supplies blood to the tumor;
introducing the substance into the blood vessel after the first embolization agent has been introduced into the blood vessel, at a position that is proximal with respect to the first embolization agent; and
introducing a second embolization agent into the blood vessel after the substance has been introduced into the blood vessel, at a position that is proximal with respect to at least a portion of the substance,
wherein the first embolization agent operates to block outflow and prevent complete systemic release of the substance, and
wherein the second embolization agent operates to block inflow and prevent retrograde washout of the substance.

2. The method of claim 1, wherein the first embolization agent comprises first embolic beads, the second embolization agent comprises second embolic beads, and the second embolic beads are larger than the first embolic beads.

3. The method of claim 2, wherein the first embolic beads have diameters less than or equal to 100 μm, and wherein the second embolic beads have diameters greater than or equal to 200 μm.

4. The method of claim 1, wherein the introducing of the first embolization agent into the blood vessel is accomplished by inflating a balloon to block the blood vessel and introducing the first embolization agent at a position that is distal with respect to the inflated balloon.

5. The method of claim 1, wherein the introducing of the substance into the blood vessel is accomplished at a pressure that is sufficient to overcome a region of high pressure in a vicinity of the tumor.

6. The method of claim 1, wherein the introducing of the substance into the blood vessel is accomplished at a pressure between 10 and 200 mmHg relative to the local pressure.

7. The method of claim 1, wherein the introducing of the substance into the blood vessel is controlled to maintain laminar flow and to prevent non target and systemic release of the sub stance.

8. The method of claim 1, wherein the substance comprises an immunotherapy sub stance.

9. The method of claim 1, wherein the substance comprises CAR T-cells.

10. A method of treating a tumor with a substance, the method comprising:
introducing a catheter into a blood vessel that supplies blood to the tumor;
inflating a balloon to block the blood vessel;
introducing the substance into the blood vessel at a position that is distal with respect to the inflated balloon at a pressure that is sufficient to overcome a region of high pressure in a vicinity of the tumor; and
introducing an embolization agent into the blood vessel after the substance has been introduced into the blood vessel, at a position that is proximal with respect to at least a portion of the substance,
wherein the embolization agent acts to prevent retrograde washout of the substance and impede systemic release by impeding the forward flow of blood.

11. The method of claim 10, wherein the embolization agent comprises embolic beads with diameters greater than or equal to 200 μm.

12. The method of claim 10, wherein the introducing of the substance into the blood vessel is accomplished at a pressure between 10 and 200 mmHg relative to the local pressure.

13. The method of claim 10, wherein the introducing of the substance into the blood vessel is controlled to maintain laminar flow and to prevent non target and systemic release of the substance.

14. The method of claim 10, wherein the substance comprises an immunotherapy sub stance.

15. The method of claim 10, wherein the substance comprises CAR T-cells.

16. A method of treating a tumor with a substance, the method comprising:
introducing a catheter into a blood vessel that supplies blood to the tumor;
inflating a balloon to block the blood vessel; and
introducing a liquid in which a plurality of substance-eluting beads are suspended into the blood vessel at a position that is distal with respect to the inflated balloon at a pressure that is sufficient to overcome a region of high pressure in a vicinity of the tumor.

17. The method of claim 16, wherein the introducing of the liquid into the blood vessel is accomplished at a pressure between 10 and 200 mmHg relative to the local pressure.

18. The method of claim 16, wherein the introducing of the liquid into the blood vessel is controlled to maintain laminar flow and to prevent non target and systemic release of the sub stance.

19. The method of claim 16, wherein the substance-eluting beads comprise an immunotherapy substance.

20. The method of claim 16, wherein the substance-eluting beads comprise CAR T-cells.

21. The method of claim 16, wherein the substance-eluting beads primarily comprise agarose.
